(19) United States
(12) Patent Application Publication
(10) Pub. No.: US 2024/0087755 A1
(43) Pub. Date: Mar. 14, 2024

(54) CREATING SYNTHETIC PATIENT DATA USING A GENERATIVE ADVERSARIAL NETWORK HAVING A MULTIVARIATE GAUSSIAN GENERATIVE MODEL

(71) Applicant: International Business Machines Corporation, Armonk, NY (US)

(72) Inventors: Daniel Enoch Platt, Putnam Valley, NY (US); Aritra Bose, White Plains, NY (US); Kahn Rhrissorrakrai, Woodside, NY (US); Aldo Guzman Saenz, White Plains, NY (US); Niina Haiminen, Tampere (FI); Laxmi Parida, Mohegan Lake, NY (US)

(21) Appl. No.: 17/930,477

(22) Filed: Sep. 8, 2022

(51) Int. Cl. G16H 50/70; G16H 50/30

(52) U.S. Cl. CPC G16H 50/70; G16H 50/30

(57) ABSTRACT

Embodiments are directed to a computer-implemented method that includes using a processor system to encode binary risk factor variables, genotypic risk factor variables, and continuous risk factor variables. The processor system is further used to adversarially train a multivariate Gaussian (MVG) generative model to generate synthetic versions of the binary risk factor variables, synthetic versions of the genotypic risk factor variables, and synthetic versions of the continuous risk factor variables.

### BACKGROUND

The present invention relates generally to programmable computers. More specifically, the present invention relates to programmable computer systems, computer-implemented methods, and computer program products operable to create multi-modal synthetic patient data using a generative adversarial network (GAN) having a multivariate Gaussian generative model.

The treatment of complex diseases requires a comprehensive understanding of the patient and the patient's history. The patient's history can be gleaned from a variety of sources, including, for example, electronic medical records; molecular profiling from whole genomic, transcriptomic, and/or proteinomic sequencing; imaging data from many time points; and the like. One goal of understanding a patient's is history is to identify disease risk factors that can assist in the diagnostic process. Risk factors are useful aids to medical diagnosis in that risk factor information is readily available to clinicians at little or no cost. It is important, however, to use risk factors having established diagnostic utility to ensure that the presence of the risk factor has an actual effect on disease probability.

Machine learning (ML) is a branch of artificial intelligence (AI) that has been used to evaluate the impact that a given risk factor has on disease probability. ML algorithms can detect patterns of certain diseases within patient electronic healthcare records and inform clinicians of any anomalies. Additionally, ML algorithms can generate predictive models that predict the influence a risk factor has on disease states. ML algorithms include three main learning modes, namely, supervised, unsupervised, and reinforcement learning. In supervised learning, a model is trained using a large volume of labeled training data (i.e., “example” data). Unsupervised learning identifies patterns in training data that are not classified or labeled then categorizes them based on the extracted features. A reinforcement learning model, in effect, trains through experience and learns to make an accurate decision based on trial and error.

Generative modeling is a type of unsupervised learning problem that automatically discovers and learns the regularities or patterns in input data in such a way that the model can be used to generate or output new examples that plausibly could have been drawn from the original dataset. Examples of unsupervised generative algorithms include generative adversarial networks (GANs) and auto-encoders (AEs) (e.g., a variational AE (VAE)).

### SUMMARY

Embodiments of the invention provide a computer-implemented method that includes using a processor system to encode binary risk factor variables, genotypic risk factor variables, and continuous risk factor variables. The processor system is further used to adversarially train a multivariate Gaussian (MVG) generative model to generate synthetic versions of the binary risk factor variables, synthetic versions of the genotypic risk factor variables, and synthetic versions of the continuous risk factor variables.

Embodiments of the invention further provide a computer system and a computer program product having substantially the same features and as the above-described computer-implemented method.

Additional features and advantages are realized through the techniques described herein. Other embodiments and aspects are described in detail herein. For a better understanding, refer to the description and to the drawings.

### BRIEF DESCRIPTION OF THE DRAWINGS

The subject matter which is regarded as the present disclosure is particularly pointed out and distinctly claimed in the claims at the conclusion of the specification. The foregoing and other features and advantages are apparent from the following detailed description taken in conjunction with the accompanying drawings in which:

FIG. 1A depicts an exemplary multivariate Gaussian GAN system in accordance with embodiments of the present invention;

FIG. 1B depicts an exemplary hybrid GAN system in accordance with embodiments of the present invention;

FIG. 2A depicts a univariate Gaussian distribution in accordance with aspects of the invention;

FIG. 2B depicts a multivariate Gaussian distribution in accordance with aspects of the invention;

FIG. 3 depicts an exemplary multivariate Gaussian generative model in accordance with aspects of the invention;

FIG. 4 depicts a diagram illustrating the relationship between chromosomes, DNA and genes in accordance with aspects of the invention;

invention;

FIG. 5 depicts genotype information in accordance with aspects of the FIG. 6 depicts a methodology in accordance with aspects of the invention;

FIG. 7A depicts a block diagram illustrating omic data sets that include real risk factor data, along with gaps in the real risk factor data;

FIG. 7B depicts a block diagram corresponding to the block diagram of FIG. 7A, where the gaps in the real risk factor data shown in FIG. 7A have been filled with synthetic risk factor data generated in accordance with aspects of the invention;

FIG. 8 depicts a machine learning system that can be utilized to implement aspects of the invention;

FIG. 9 depicts a learning phase that can be implemented by the machine learning system shown in FIG. 8; and

FIG. 10 depicts a computing environment capable of implementing aspects of the invention.

In the accompanying figures and following detailed description of the disclosed embodiments, the various elements illustrated in the figures are provided with three or four digit reference numbers. The leftmost digit(s) of each reference number corresponds to the figure in which its element is first illustrated.

### DETAILED DESCRIPTION

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Many of the functional units described in this specification are illustrated as logical blocks such as generators, discriminators, modules, processors, and the like. Embodiments of the invention apply to a wide variety of implementations of the logical blocks described herein. For example, a given logical block can be implemented as a hardware circuit operable to include custom VLSI circuits or gate arrays, as well as off-the-shelf semiconductors such as logic chips, transistors, or other discrete components. The logical blocks can also be implemented in programmable hardware devices such as field programmable gate arrays, programmable array logic, programmable logic devices, and the like. The logical blocks can also be implemented in software for execution by various types of processors. Some logical blocks described herein can be implemented as one or more physical or logical blocks of computer instructions which can, for instance, be organized as an object, procedure, or function. The executables of a logical block described herein need not be physically located together but can include disparate instructions stored in different locations which, when joined logically together, include the logical block and achieve the stated purpose for the logical block.

Turning now to a more detailed description of technologies that are relevant to aspects of the invention, as previously noted herein, the treatment of complex diseases requires a comprehensive understanding of the patient and the patient's history. The patient's history can be gleaned from a variety of sources, including, for example, electronic medical records; molecular profiling from whole genomic, transcriptomic, and/or proteinomic sequencing; imaging data from many time points; and the like. One goal of understanding a patient's is history is to identify disease risk factors that can assist in the diagnostic process. Risk factors are useful aids to medical diagnosis in that risk factor information is readily available to clinicians at little or no cost. It is important, however, to use risk factors having established diagnostic utility to ensure that the presence of the risk factor has an actual effect on disease probability.

Cancer is an example of a highly complex disease with a complex etiology rooted in the genome of the cell. As such, cancer analysis and diagnosis benefits from a deep characterization of its omic profile. The branches of science known informally as “omics” are various disciplines in biology whose names end in the suffix “omics,” such as genomics, proteomics, metabolomics, metagenomics, phenomics and transcriptomics. Omics aims at the collective characterization and quantification of pools of biological molecules that translate into the structure, function, and dynamics of an organism or organisms. Thus, a variety of technologies and informatics systems have been developed that generate and process large biological data sets (i.e., omics data). In healthcare, informatics systems use various types of information technology to organize and analyze health records to improve healthcare outcomes. “Health” informatics systems deal with the resources, devices, and methods required to acquire, store, retrieve, and use health and medical data.

Because single oncogenic and resistant driver genes explain only a fraction of all cancers, capturing events that phenocopy these drivers necessitates analyzing other modalities (or types) of data that offer different types of information, including, for example, genome sequencing, RNA sequencing, clinical medical records, clinical assays, and the like. However, access to the medical data needed to create the above-described datasets is often limited due to a variety of factors such as privacy laws, health industry standards, the lack of integration of medical information systems, and other considerations. As a result, incompleteness is present in each of these above-described datasets for any given patient. In some instances, entire modes of data can be missing from blocks in the dataset.

Data gaps can be filled by using neural networks such as GANs to generate so-called synthetic data. Synthetic data is artificially created data that is designed to replicate the statistical characteristics and correlations of real-world, raw data. However, known systems for generating synthetic data are complicated, computationally expensive, and produce their synthetic data through complicated functional input/output relationships. Accordingly, the use of known neural network systems to generate synthetic data for medical diagnosis/analysis applications would not uncover direct and easily-understood correlations between risk factors and disease states, particularly for multi-modal data and analysis. Thus, known synthetic data generation systems do not generate synthetic data that is sufficiently representative of a specific patient to be biologically relevant; do not uncover input/output (i.e., risk-factor/disease-state) relationships and characteristics from which meaningful insights can be derived; and do not enable the ability to develop the comprehensive understanding of patients and patient histories that is necessary for the accurate diagnosis and treatment of complex diseases.

Turning now to an overview of aspects of the present invention, embodiments of the invention provide programmable computer systems, computer-implemented methods, and computer program products operable to create multi-modal synthetic patient data using a novel multivariate Gaussian GAN (MVG-GAN) having a multivariate Gaussian (MVG) generative model. In embodiments of the invention, the MVG-GAN trains its MVG generative model by framing the problem as a supervised learning problem with two sub-models, namely the MVG generative model and a discriminative model. The MVG generative model is trained to generate multi-modal examples in a multivariate Gaussian distribution, and the discriminative model tries to classify the examples as either real (i.e., from the multivariate Gaussian domain) or fake (i.e., generated or non-authentic). The MVG generative model and the discriminative model are trained together in an adversarial zero-sum game until the discriminative model is fooled about half the time, which means the MVG generative model is generating plausible examples that the discriminator model cannot identify as fake. In this detailed description, generative model examples that do not fool the discriminative model are referred to as fake examples; and generative model examples that fool the discriminative model quality as synthetic examples.

In embodiments of the invention, the novel multivariate Gaussian GAN is multi-modal in that it creates a multivariate Gaussian distribution from risk factor (RF) variables encoded into three major modalities or categories, which are defined herein as binary RF variables, genotypic RF variables, and continuous RF variables. In embodiments of the invention, binary RF variables identify risk factors that are either present or not present, examples of which include the various individual disease states of metabolic syndrome. In embodiments of the invention, genotypic RF variables identify risk factors that are reflected in the patient's genotype. A gene is a locus or region of DNA that is the molecular unit of heredity. Genes are made up of molecules inside the nucleus of a cell that are strung together in such a way that the sequence carries information. This information determines how living organisms inherit phenotypic traits (i.e., features), which are determined by the genes they received from their parents, grandparents and so on, going back through generations. Most biological traits are under the influence of many different genes, as well as gene—environment interactions. Some genetic traits are instantly visible, such as eye color or number of limbs, and some are not, such as blood type, risk for specific diseases, or any one of the thousands of basic biochemical processes that comprise life. An organism's genotype is the internally coded, inheritable information carried by all living organisms. Genotype information is used as a “blueprint” or set of instructions for building and maintaining a living creature. These instructions are found within almost all cells and are they are written in a coded language known generally as the “genetic code.” Genetic code instructions are copied at the time of cell division or reproduction (i.e., meiosis) and are passed from one generation to the next through inheritance. Genetic code instructions are intimately involved with all aspects of the life of a cell or an organism. They control everything from the formation of protein macromolecules to the regulation of metabolism and synthesis. In embodiments of the invention, continuous RF variables identify risk factors that are present along a continuum, examples of which include gene expression data, quantitative traits, or how much of a particular drug a patient is taking.

Accordingly, the MVG-GAN avoids the shortcomings of known systems for generating synthetic data by incorporating an MVG generator that generates synthetic data that is sufficiently representative of a specific patient to be biologically relevant; that uncovers input/output (i.e., risk-factor/disease-state) relationships and characteristics from which meaningful insights can be derived; and that enables the ability to develop the comprehensive understanding of patients and patient histories that is necessary for the accurate diagnosis and treatment of complex diseases.

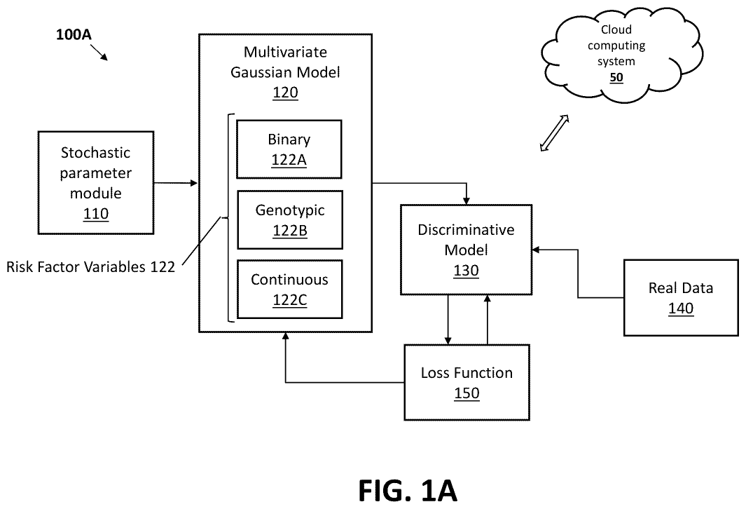

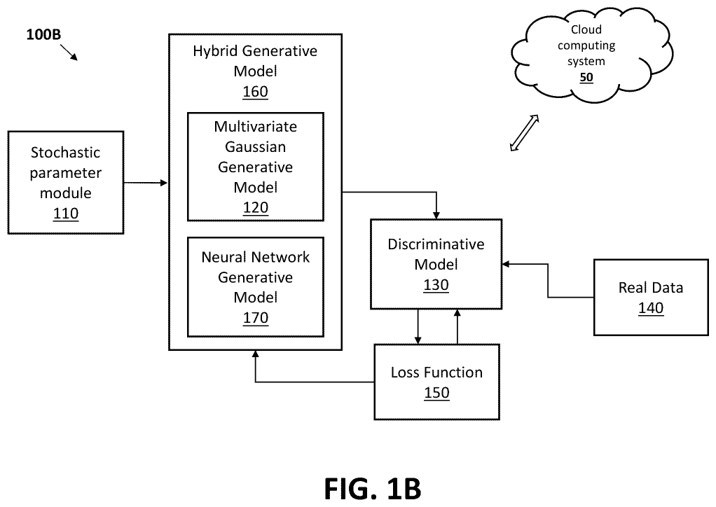

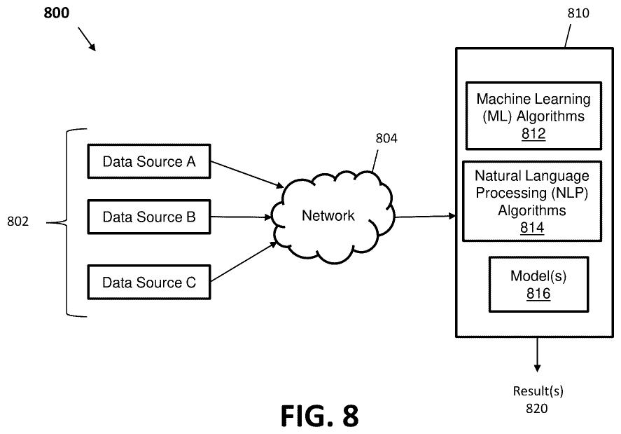

Turning now to a more detailed description of aspects of the present invention, FIG. 1A is a non-limiting, simplified block diagram of an MVG-GAN system 100A having an MVG generative model 120 in accordance with embodiments of the invention. More specifically, the system 100A includes a stochastic parameter module 110, the MVG generative model 120, a discriminative model 130, a real data module 140, and a loss function module 150, configured and arranged as shown. In embodiments of the invention, the untrained version of the MVG generative model 120 is operable to begin the process of generating fake RF variables 122 in an MVG distribution. The discriminative model 130 can be implemented as a classifier (e.g., classifier 810 shown in FIG. 8) trained to distinguish fake RF variables 122 from real RF variables generated by the real data module 140.

In embodiments of the invention, a cloud computing system 50 is in wired or wireless communication with one or more components/modules of the system 100A. Cloud computing system 50 can supplement, support, or replace some or all of the functionality of the components/modules of the system 100A. Additionally, some or all of the functionality of the components/modules that form the system 100A can be implemented as a node of the cloud computing system 50.

The various components/modules of the system 100A shown in FIG. 1A are depicted separately for ease of illustration and explanation. In embodiments of the invention, the functions performed by the various components/modules of the system 100A can be distributed differently than shown. For example, in some embodiments of the invention, the MVG generative model 120 could be integrated into the discriminative model 130 and vice versa.

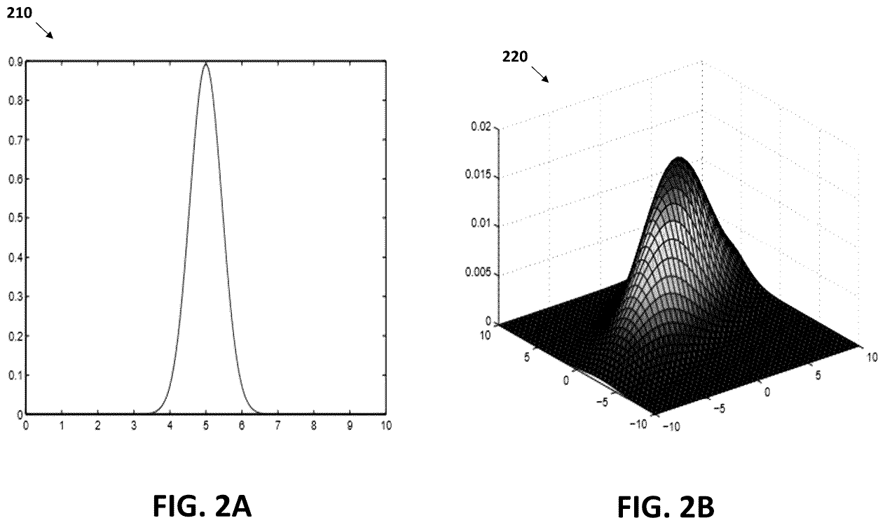

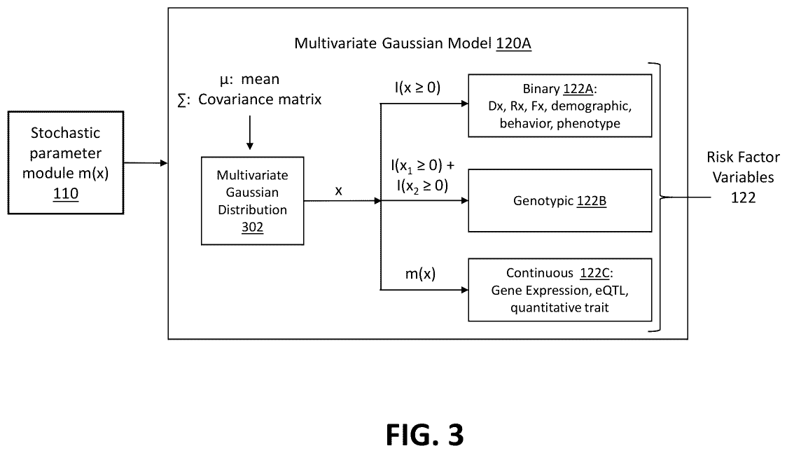

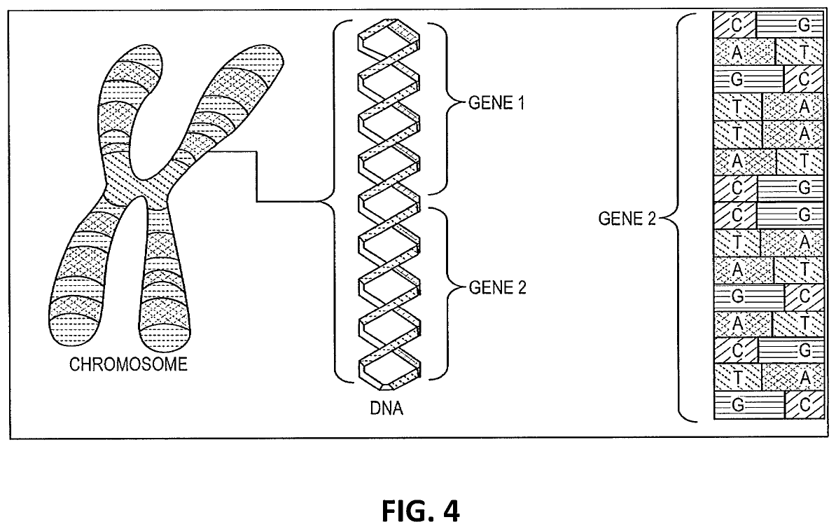

The multivariate Gaussian distribution (e.g., the multivariate Gaussian distribution 302 shown in FIG. 3) used in the MVG generative model 120 (shown in FIG. 1A) is a multidimensional generalization of the one-dimensional (or univariate) Gaussian (or normal) distribution. FIG. 2A depicts a univariate Gaussian distribution 210; and FIG. 2B depicts a multivariate Gaussian distribution 220 in accordance with aspects of the invention. The multivariate Gaussian distribution 220 is bivariate for ease of illustration. However, in accordance with aspects of the invention, the multivariate Gaussian distribution 220 can include higher dimensions (more than two (2) variables). In general, a multivariate is a vector with each of its elements being a variate. The variates need not be independent, and if they are not, a correlation is said to exist between them. The term “multivariate” is also used as an adjective to mean involving many variables, as opposed to one (univariate) or two (bivariate). In comparison to the multivariate Gaussian distribution 220, the one-dimensional Gaussian distribution 210 has a two-dimensional (2D) bell shape, and is one of the most common in all of statistics. The “central limit theorem” demonstrates that sums of large numbers of independent, identically distributed random variables are well approximated by a Gaussian distribution. The parameter estimates in a statistical model are also asymptotically Gaussian. Gaussians are widely used in probabilistic modeling for these reasons, together with the fact that Gaussian distributions can be efficiently manipulated using the techniques of linear algebra. The parameters of an n-dimension multivariate Gaussian distribution are an n-dimensional mean vector and an n-by-n dimensional covariance matrix. In other words, the multivariate Gaussian distribution 220 represents the distribution of a multivariate that is made up of multiple random variables that can be correlated with each other. In probability, and in statistics, a multivariate random variable or random vector is a list of mathematical variables each of whose value is unknown, either because the value has not yet occurred or because there is imperfect knowledge of its value. The multivariate Gaussian distribution 220 is defined by sets of parameters, namely the mean vector μ, which is the expected value of the distribution; and the covariance matrix Σ, which measures how dependent the random variables are and how they change together. Additional details of the how the MVG generative model 120 can be implemented as an MVG generative model 120A are depicted in FIG. 3 and described in greater details subsequently herein.

Referring again to FIG. 1A, in accordance with aspects of the invention, the RF variables 122 are vectorized and encoded in any suitable fashion that allows them to be analyzed by the MGV generative model 120 and organized in an MVG distribution (e.g., MVG distribution 220 shown in FIG. 2A). In accordance with aspects of the invention, the RF variables 122 include binary RF variables 122A, genotypic RF variables 122B, and continuous RF variables 122C. In embodiments of the invention, the binary RF variables 122A identify risk factors that are either present or not present, examples of which include the various individual disease states of metabolic syndrome. In embodiments of the invention, genotypic RF variables 122B identify risk factors that are reflected in the patient's diploid single nucleotide polymorphism genotypes. A gene is a locus or region of DNA that is the molecular unit of heredity. Genes are made up of molecular sequences of nucleotides inside the nucleus of a cell that are strung together in such a way that the cell's biochemistry can decode the sequence into protein coding information. The nucleotide polymorphism information determines how living organisms inherit phenotypic traits (i.e., features), which are determined by the genes they received from their parents, grandparents and so on, going back through generations. Most biological traits are under the influence of many different genes, as well as gene—environment interactions. Some genetic traits are instantly visible, such as eye color or number of limbs, and some are not, such as blood type, risk for specific diseases, or any one of the thousands of basic biochemical processes that comprise life. An organism's genotype is the internally coded, inheritable information carried by all living organisms. Genotype information is used as a “blueprint” or set of instructions for building and maintaining a living creature. These instructions are found within almost all cells and are they are written in a coded language known generally as the “genetic code.” Genetic code instructions are copied at the time of cell division or reproduction (i.e., meiosis) and are passed from one generation to the next through inheritance. Genetic code instructions are intimately involved with all aspects of the life of a cell or an organism. They control everything from the formation of protein macromolecules to the regulation of metabolism and synthesis. Single nucleotide polymorphisms associate with traits, such as disease, among population members. In embodiments of the invention, continuous RF variables 122C identify risk factors that are present along a continuum, examples of which include gene expression data, quantitative traits, or behavior-type information such as how much of a particular drug a patient is taking or how often a patient exercises.

As shown in FIG. 1A, the MVG generative model 120 takes stochastic vectors and transforms the stochastic vectors to mimic an MVG distribution of the risk factor variables 122 (i.e., the binary RF variables 122A, the genotypic RF variables 122B, and the continuous RF variables 122C). Batches of the generated (fake) risk factor variables 122 from the MVG generative model 120, along with real data (i.e., real risk factor variables) from the real data module 140, are sent to the discriminative model 130, where the discriminative model 130 assigns a label of zero (0) for “real” or a label of one (1) for “fake.” After an iteration of data is passed through both the MVG generative model 120 and the discriminative model 130, the loss function module 150 provides learning feedback to the MVG generative model 120 and the discriminative model 130 so the models 120, 130 can continue improving at a level pace, which means that the MVG generative model 120 continues to try to outsmart the discriminative model 130 by generating better fakes, and also means that the discriminative model 130 continues to make a correct classification of both the real and fake input so that the MVG generative model 120 can keep getting better. Eventually, equilibrium is reached when the MVG generative model 120 outputs risk factors that look real enough to be part of the original real data set 140 that you use to train the discriminative model 130, which means the generated data can now be characterized as synthetic data. The equilibrium point can be exactly when the discriminative model 130 is leaning 50% to both sides, meaning that both sets of risk factors (one set from the MVG generative model 120 and one set from the real data 140) could either be real or fake. This means that the MVG generative model 120 tries to minimize the probability that the discriminative model 130 will predict the MVG generative model output as fake. Conversely, the discriminative model 130 tries to maximize the probability that it will correctly classify both real risk factors and fake risk factors. With an appropriate optimization technique in the loss function module 150, the neural network of the discriminative model 130 and the MVG generative model 120 can be trained to reach an optimal point where the MVG generative model 120 produces realistic or synthetic RF data 122 and the optimal discriminative model 130 will estimate the likelihood of a given synthetic RF data set being real.

FIG. 1B is a non-limiting, simplified block diagram of an MVG-GAN system 100B having a hybrid MVG generative model 160 in accordance with embodiments of the invention. The system 100B is substantially the same as the system 100A (shown in FIG. 1A) except the MVG generative model 120 of the system 100A is replaced with the hybrid generative model 160 having the MVG generative model 120 and a neural network generative model 170. The MVG generative model 120 in system 100B is operable to produce synthetic RF variables 122 in substantially the same manner as it does in the system 100A; and the synthetic RF variables 122 produced in system 100B are used by the neural network generative model 170, the discriminative model 130, the real data module 140, and the loss function module 150 to augment the performance of GAN-type operations such as generating synthetic image data (e.g., synthetic X-ray image data) to augment image data (e.g., X-ray image data) used to assist clinicians in diagnosing disease states.

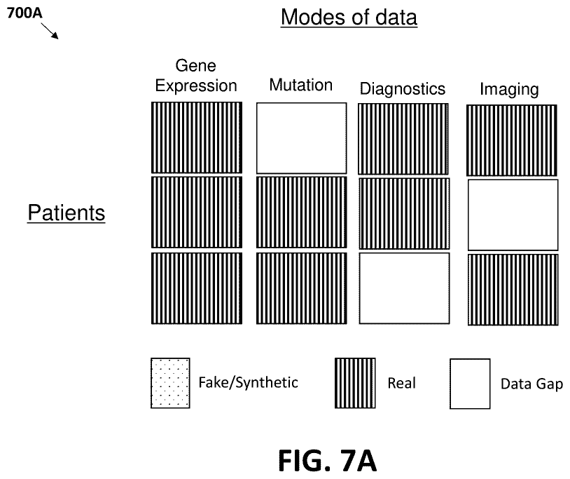

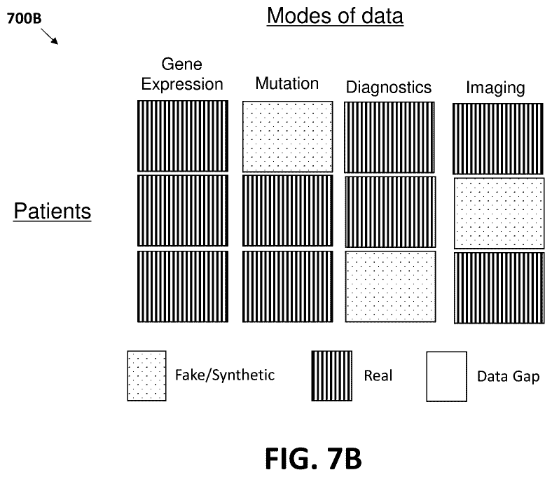

An example of results generated by the system 100B is depicted by the block diagrams 700A, 700B shown in FIGS. 7A and 7B, respectively. Block diagram 700A illustrates omic data sets that include real risk factor data, along with gaps in the real risk factor data. Block diagram 700B corresponds to the block diagram 700A except that the gaps in the real risk factor data shown block diagram 700A have been filled in block diagram 700B with synthetic risk factor data generated in accordance with aspects of the invention. Referring to FIGS. 7A and 7B, gene expression data corresponds to continuous RF variables 122C; mutations data corresponds to genotypic RF variables 122B; and diagnosis data corresponds to binary RF variables 122A. The MVG generative model 120, the discriminative model 130, the real data module 140, and the loss function module 150 shown in FIG. 1B operate as previously described to generate the synthetic RF variable data that fills the gaps under mutation data and diagnostics data (shown in FIGS. 7A and 7B). The system 100B then uses the gene expression data, the mutation data (with data gaps filled), and the diagnostics data (with data gaps filled) as part of the real data module 140, which is used by the neural network generative model 170, the discriminative model 130 and the loss function model 150 to generate synthetic image data to fill out gaps in the image data as shown in the diagram 700B.

FIG. 3 depicts details of the how the MVG generative model 120 (shown in FIGS. 1A and 1B) of the system 100A, 100B (shown in FIGS. 1A and 1B) can be implemented as an MVG generative model 120A in accordance with aspects of the invention. As shown in FIG. 3, the MVG generative model 120A includes an n-dimensional (n≥2; and/or n>2) multivariate Gaussian distribution 302, and the system 100A, 100B trains the n-dimensional MVG generative model 120A (in the manner depicted in FIGS. 1A and 1B) to generate fake versions of the risk factor variables 122 and fit them into the n-dimensional multivariate Gaussian distribution 302. The parameters of the n-dimension multivariate Gaussian distribution 302 are an n-dimensional mean vector and an n-by-n dimensional covariance matrix. In other words, the n-dimensional multivariate Gaussian distribution 302 represents the distribution of a multivariate that is made up of multiple random variables (the risk factor variables 122) that can be correlated with each other. In probability, and in statistics, a multivariate random variable or random vector is a list of mathematical variables each of whose value is unknown, either because the value has not yet occurred or because there is imperfect knowledge of its value.

The n-dimensional multivariate Gaussian distribution 302 is defined by sets of parameters, namely the mean vector μ, which is the expected value of the distribution; the covariance matrix Σ, which measures how dependent the random variables are and how they change together; and a user-specified map m(x), which maps sigmoid distributions of individual output variable to other distributions, thereby taking the output to another cumulative distribution.

Similar to FIG. 1A, the RF variables 122 in FIG. 3 are vectorized and encoded in any suitable fashion that allows them to be analyzed by the MGV generative model 120A and organized in an MVG distribution 302. In accordance with aspects of the invention, the RF variables 122 include binary RF variables 122A, genotypic RF variables 122B, and continuous RF variables 122C. In embodiments of the invention, the binary RF variables 122A identify risk factors that are either present or not present, examples of which include the various individual disease states of metabolic syndrome. In the example depicted in FIG. 3, the binary RF variables 122A include Dx (e.g., a diagnosis), Rx (e.g., a prescription), Fx (e.g., family history), demographic (e.g., traditional diet, behavior, prohibitions against alcohol, and other cultural traits that impact risk factors), behavior (e.g., exercise, diet, and risky activities), and phenotype (e.g., physical manifestations of genetic variants). In embodiments of the invention, genotypic RF variables 122B identify risk factors that are reflected in the patient's diploid single nucleotide polymorphism genotypes. In embodiments of the invention, continuous RF variables 122C identify risk factors that are present along a continuum, examples of which include gene expression data, quantitative traits (e.g., height, floating point numbers such as blood pressure, LDL levels, triglyceride levels, etc.), and eQTL (“expression quantitative trait loci”). eQTL is how a single SNP impacts the amount of protein that is being expressed from DNA.

Operation of the MVG generative model 120A in the context of the systems 100A, 100B will now be provided with reference to a computer-implemented methodology 600 shown in FIG. 6. Methodology 600 starts at block 602 then moves in parallel to blocks 604, 606, 608. At block 604, encoding is defined for the binary RF variables 122A. The encoding can be any suitable format for the MVG distribution 302. In some embodiments of the invention, the encoding of the binary RF variables 122A can be represented by the notation “I(x≥0)”. I(S) is one (1) if statement S is true, and I(S) is zero otherwise. Thus, if, for example, a gaussian variate has a probability of 0.66 of being positive, then *(c≥0)′ has a value of one (1) 0.66 of the time. Accordingly, as a mapping function, it can take a continuous Gaussian variable and map it to a binary Bernoulli distribution. At block 606, encoding is defined for the genotypic RF variables 122B. The encoding can be any suitable format for the MVG distribution 302. In some embodiments of the invention, the encoding of the genotypic RF variables 122B can be represented by the notation “I(x₁≥0)+I(x₂≥0)”. The sum of I(x₁≥0)+I(x₂≥0) represents the sum of two Bernoulli binary variates. These represent the single nucleotide polymorphism values from the two DNA (diploid) strands. Such a sum is binomially distributed with n=2. At block 608, encoding is defined for the continuous RF variables 122C. The encoding can be any suitable format for the MVG distribution 302. In some embodiments of the invention, the encoding of the continuous RF variables 122C can be represented by the notation “m(x)”, where m(x) is the map from a Gaussian distributed x to continuous, binary, and n=2 binomial diploid SNP data representations. It is how multimodal data are generated from an underlying Gaussian.

The methodology 600 then move to block 610 where parameters of the MVG generative model 120A are defined, and the MVG parameters, the binary RF variables 122A, the genotypic RF variables 122B, and the continuous RF variables 122C are loaded into the MVG generative model 120A having the MVG distribution 302. At block 612, the system 100A, 100B uses the discriminative model 140, the real data module 140, and the loss function module 150 to adversarially train the MVG generative model 120A to generate synthetic versions of the binary RF variables 122A, synthetic versions of the genotypic RF variables 122b, and synthetic versions of the continuous RF variables 122C in the MVG distribution 302. The methodology 600 moves in parallel from block 612 to blocks 614 and 616. At block 614, the system 100A, 100B extracts the synthetic versions of the binary RF variables 122A, the synthetic versions of the genotypic RF variables 122b, and the synthetic versions of the continuous RF variables 122C, which can all be provided to other omic data analysis systems to fill in omic data gaps (e.g., as shown in FIGS. 7A and 7B) and improve overall omic data analysis and disease diagnosis operations performed by such omic data analysis systems. At block 616, the system 100A, 100B extracts correlations between and among the synthetic versions of the binary RF variables 122A, the synthetic versions of the genotypic RF variables 122b, and the synthetic versions of the continuous RF variables 122C. The correlations (as well as the synthetic versions 122A, 122B, 122C) can be provided to other omic data analysis systems to fill in omic data gaps (e.g., as shown in FIGS. 7A and 7B) and improve overall omic data analysis and disease diagnosis operations performed by such omic data analysis systems.

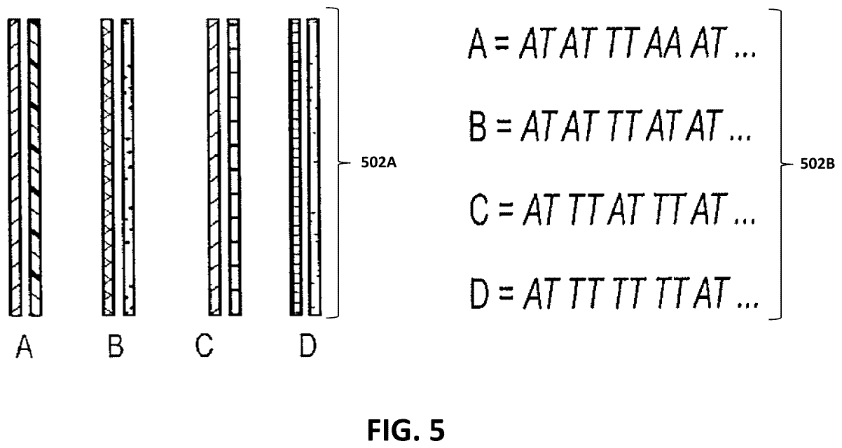

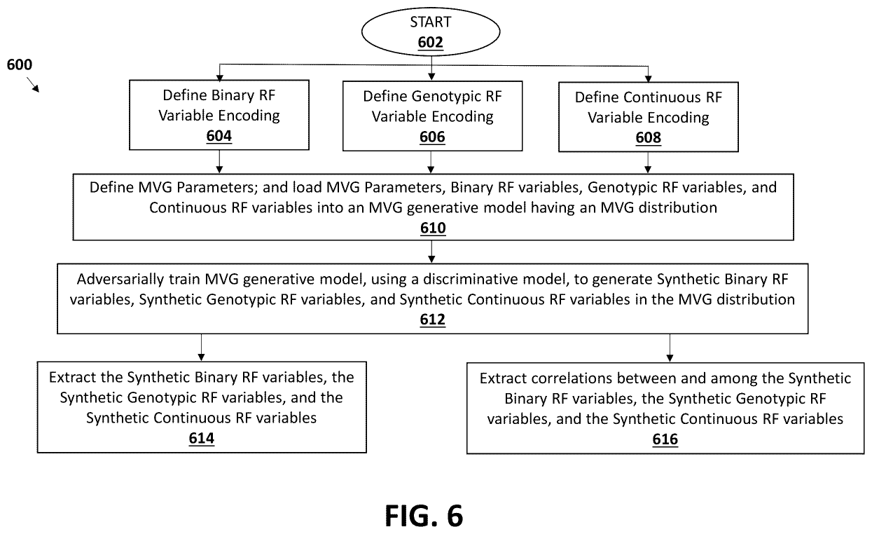

FIGS. 4 and 5 provide additional details of the nature of genotypic RF variables 122B that can be used and/or analyzed in aspects of the invention. The relationship between chromosomes, DNA and genes is shown in FIG. 4. The chromosomes of a cell are in the cell nucleus. Chromosomes contain many genes and carry the genetic information of the organism. Chromosomes are made up of DNA and protein combined as chromatin. All animal cells have a fixed number of chromosomes in their body cells, which exist in homologous pairs. Each chromosome pair is described as a diploid, and each individual chromosome is described as a haploid.

Different animals have different numbers of chromosomes. For example, there are 23 chromosome pairs (i.e., 46 in total) in a human, including a pair of sex hormones. Human progeny receives a set of 23 chromosomes from their father and a matching set of 23 chromosomes from their mother. To produce each parent's 23 sex cells (gametes) for donation to the progeny, the stem cells go through a different division process called meiosis, which reduces the parent's 23 chromosome pairs (i.e., diploids) to 23 individual chromosomes (i.e., haploids), which combine with the other parent's 23 pair through fertilization to produce the new set of 23 pairs of the progeny.

The terms homozygous, heterozygous and hemizygous are used to describe the genotype of a diploid organism at a single locus on the DNA. Homozygous describes a genotype consisting of two identical alleles at a given locus, and heterozygous describes a genotype consisting of two different alleles at a locus. Hemizygous describes a genotype consisting of only a single copy of a particular gene in an otherwise diploid organism.

Analysis of risk factors for a given disease requires extensive study and analysis of an organism's genotype, which is the internally coded, inheritable information carried by all living organisms. Genotype information is used as a “blueprint” or set of instructions for building and maintaining a living creature. These instructions are found within almost all cells and they are written in a coded language known generally as the “genetic code.” Genetic code instructions are copied at the time of cell division or reproduction (i.e., meiosis) and are passed from one generation to the next through inheritance. Genetic code instructions are intimately involved with all aspects of the life of a cell or an organism. They control everything from the formation of protein macromolecules to the regulation of metabolism and synthesis.

FIG. 5 depicts an example of a set of genotypes 502A of a set of patients A, B, C, D. The genotypes 502A of patients A, B, C, D also include chromosome pairs, which are shown in FIG. 5 as vertical bars. FIG. 5 also depicts a genotype 502B of the set of patients A, B, C, D represented as a DNA sequence of single nucleotides—A, T, C, or G. A single nucleotide polymorphism, also known as a simple nucleotide polymorphism, (SNP) is a DNA sequence variation occurring commonly within a population (e.g. 1%) in which a single nucleotide (A, T, C or G) in the genome (or other shared sequence) differs between members of a biological species or paired chromosomes. For example, two sequenced DNA fragments from different individuals, AAGCCTA to AAGCTTA, contain a difference in a single nucleotide. In this case we say that there are two alleles. Almost all common SNPs have only two alleles. The genomic distribution of SNPs is not homogenous. SNPs occur in non-coding regions more frequently than in coding regions or, in general, where natural selection is acting and fixating the allele (eliminating other variant) of the SNP that constitutes the most favorable genetic adaptation. Other factors, like genetic recombination and mutation rate, can also determine SNP density.

There are variations between human populations, so a SNP allele that is common in one geographical or ethnic group may be much rarer in another. These genetic variations between individuals (particularly in non-coding parts of the genome) underlie differences in our susceptibility to disease. The severity of illness and the way our body responds to treatments are also manifestations of genetic variations. For example, a single base mutation in the APOE (apolipoprotein E) gene is associated with a higher risk for Alzheimer's disease. Variations in the DNA sequences of humans can also affect how humans develop diseases and respond to pathogens, chemicals, drugs, vaccines, and other agents. SNPs are also critical for personalized medicine. However, their greatest importance in biomedical research is for comparing regions of the genome between cohorts (such as with matched cohorts with and without a disease) in genome-wide association studies.

Accordingly, it can be seen from the foregoing detailed description that embodiments of invention provide technical benefits and create technical effects. Embodiments of the invention provide programmable computer systems, computer-implemented methods, and computer program products operable to create multi-modal synthetic patient data using a novel MVG-GAN having a novel MVG generative model. In embodiments of the invention, the MVG-GAN adversarially trains its MVG generative model to generate multi-modal synthetic examples in a multivariate Gaussian distribution. The novel multivariate Gaussian GAN is multi-modal in that it creates a multivariate Gaussian distribution from RF variables encoded into three major modalities or categories, which are defined herein as binary RF variables, genotypic RF variables, and continuous RF variables. In embodiments of the invention, binary RF variables identify risk factors that are either present or not present, examples of which include the various individual disease states of metabolic syndrome. In embodiments of the invention, genotypic RF variables identify risk factors that are reflected in the patient's genotype. In embodiments of the invention, continuous RF variables identify risk factors that are present along a continuum, examples of which include gene expression data, quantitative traits, or how much of a particular drug a patient is taking.

The novel MVG generative model, once trained, is operable to generate synthetic versions of the binary RF variables, the synthetic versions of the genotypic RF variables, and synthetic versions of the continuous RF variables 122C, which can all be provided to other omic data analysis systems to fill in omic data gaps and improve overall omic data analysis and disease diagnosis operations performed by such omic data analysis systems. The trained MVG generative model is further operable to generate correlations between and among the synthetic versions of the binary RF variables, the synthetic versions of the genotypic RF variables, and the synthetic versions of the continuous RF variables. The correlations, as well as the synthetic versions of the RF variables, can be provided to other omic data analysis systems to fill in omic data gaps and improve overall omic data analysis and disease diagnosis operations performed by such omic data analysis systems.

Accordingly, the MVG-GAN avoids the shortcomings of known systems for generating synthetic data by incorporating an MVG generative model that generates synthetic data that is sufficiently representative of a specific patient to be biologically relevant; that uncovers input/output (i.e., risk-factor/disease-state) relationships and characteristics from which meaningful insights can be derived; and that enables the ability to develop the comprehensive understanding of patients and patient histories that is necessary for the accurate diagnosis and treatment of complex diseases.

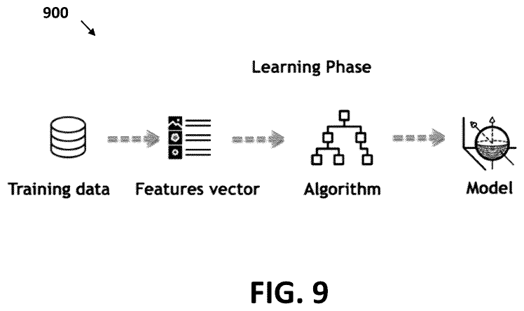

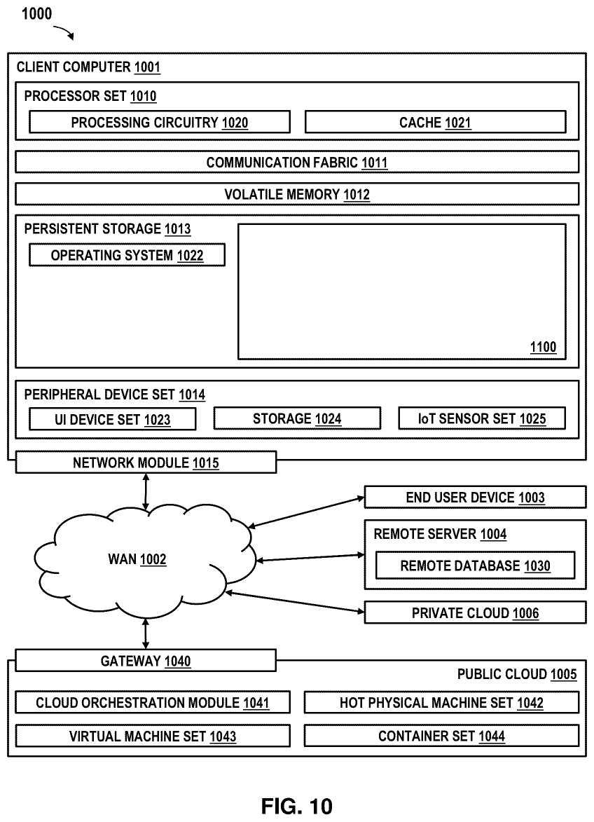

An example of machine learning techniques that can be used to implement aspects of the invention will be described with reference to FIGS. 8 and 9. Machine learning models configured and arranged according to embodiments of the invention will be described with reference to FIG. 8. Detailed descriptions of an example computing environment 1000 and network architecture capable of implementing embodiments of the invention described herein will be provided with reference to FIG. 10.

FIG. 8 depicts a block diagram showing a classifier system 800 capable of implementing various aspects of the invention described herein. More specifically, the functionality of the system 800 is used in embodiments of the invention to generate various models and/or sub-models that can be used to implement computer functionality in embodiments of the invention. The system 800 includes multiple data sources 802 in communication through a network 804 with a classifier 810. In some aspects of the invention, the data sources 802 can bypass the network 804 and feed directly into the classifier 810. The data sources 802 provide data/information inputs that will be evaluated by the classifier 810 in accordance with embodiments of the invention. The data sources 802 also provide data/information inputs that can be used by the classifier 810 to train and/or update model(s) 816 created by the classifier 810. The data sources 802 can be implemented as a wide variety of data sources, including but not limited to, sensors configured to gather real time data, data repositories (including training data repositories), and outputs from other classifiers. The network 804 can be any type of communications network, including but not limited to local networks, wide area networks, private networks, the Internet, and the like.

The classifier 810 can be implemented as algorithms executed by a programmable computer such as the computing environment 1000 (shown in FIG. 10). As shown in FIG. 8, the classifier 810 includes a suite of machine learning (ML) algorithms 812; natural language processing (NLP) algorithms 814; and model(s) 816 that are relationship (or prediction) algorithms generated (or learned) by the ML algorithms 812. The algorithms 812, 814, 816 of the classifier 810 are depicted separately for ease of illustration and explanation. In embodiments of the invention, the functions performed by the various algorithms 812, 814, 816 of the classifier 810 can be distributed differently than shown. For example, where the classifier 810 is configured to perform an overall task having sub-tasks, the suite of ML algorithms 812 can be segmented such that a portion of the ML algorithms 812 executes each sub-task and a portion of the ML algorithms 812 executes the overall task. Additionally, in some embodiments of the invention, the NLP algorithms 814 can be integrated within the ML algorithms 812.

The NLP algorithms 814 includes text recognition functionality that allows the classifier 810, and more specifically the ML algorithms 812, to receive natural language data (e.g., text written as English alphabet symbols) and apply elements of language processing, information retrieval, and machine learning to derive meaning from the natural language inputs and potentially take action based on the derived meaning. The NLP algorithms 814 used in accordance with aspects of the invention can also include speech synthesis functionality that allows the classifier 810 to translate the result(s) 820 into natural language (text and audio) to communicate aspects of the result(s) 820 as natural language communications.

The NLP and ML algorithms 814, 812 receive and evaluate input data (i.e., training data and data-under-analysis) from the data sources 802. The ML algorithms 812 include functionality that is necessary to interpret and utilize the input data's format. For example, where the data sources 802 include image data, the ML algorithms 812 can include visual recognition software configured to interpret image data. The ML algorithms 812 apply machine learning techniques to received training data (e.g., data received from one or more of the data sources 802) in order to, over time, create/train/update one or more models 816 that model the overall task and the sub-tasks that the classifier 810 is designed to complete.

Referring now to FIGS. 8 and 9 collectively, FIG. 9 depicts an example of a learning phase 900 performed by the ML algorithms 812 to generate the above-described models 816. In the learning phase 900, the classifier 810 extracts features from the training data and converts the features to vector representations that can be recognized and analyzed by the ML algorithms 812. The feature vectors are analyzed by the ML algorithm 812 to “classify” the training data against the target model (or the model's task) and uncover relationships between and among the classified training data. Examples of suitable implementations of the ML algorithms 812 include but are not limited to neural networks, support vector machines (SVMs), logistic regression, decision trees, hidden Markov Models (HMMs), etc. The learning or training performed by the ML algorithms 812 can be supervised, unsupervised, or a hybrid that includes aspects of supervised and unsupervised learning. Supervised learning is when training data is already available and classified/labeled. Unsupervised learning is when training data is not classified/labeled so must be developed through iterations of the classifier 810 and the ML algorithms 812. Unsupervised learning can utilize additional learning/training methods including, for example, clustering, anomaly detection, neural networks, deep learning, and the like.

When the models 816 are sufficiently trained by the ML algorithms 812, the data sources 802 that generate “real world” data are accessed, and the “real world” data is applied to the models 816 to generate usable versions of the results 820. In some embodiments of the invention, the results 820 can be fed back to the classifier 810 and used by the ML algorithms 812 as additional training data for updating and/or refining the models 816.

In aspects of the invention, the ML algorithms 812 and the models 816 can be configured to apply confidence levels (CLs) to various ones of their results/determinations (including the results 820) in order to improve the overall accuracy of the particular result/determination. When the ML algorithms 812 and/or the models 816 make a determination or generate a result for which the value of CL is below a predetermined threshold (TH) (i.e., CL<TH), the result/determination can be classified as having sufficiently low “confidence” to justify a conclusion that the determination/result is not valid, and this conclusion can be used to determine when, how, and/or if the determinations/results are handled in downstream processing. If CL>TH, the determination/result can be considered valid, and this conclusion can be used to determine when, how, and/or if the determinations/results are handled in downstream processing. Many different predetermined TH levels can be provided. The determinations/results with CL>TH can be ranked from the highest CL>TH to the lowest CL>TH in order to prioritize when, how, and/or if the determinations/results are handled in downstream processing.

In aspects of the invention, the classifier 810 can be configured to apply confidence levels (CLs) to the results 820. When the classifier 810 determines that a CL in the results 820 is below a predetermined threshold (TH) (i.e., CL<TH), the results 820 can be classified as sufficiently low to justify a classification of “no confidence” in the results 820. If CL>TH, the results 820 can be classified as sufficiently high to justify a determination that the results 820 are valid. Many different predetermined TH levels can be provided such that the results 820 with CL>TH can be ranked from the highest CL>TH to the lowest CL>TH.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

FIG. 10 depicts an example computing environment 1000 that can be used to implement aspects of the invention. Computing environment 1000 contains an example of an environment for the execution of at least some of the computer code involved in performing the inventive methods, such as an improved generative adversarial network having a novel multivariate Gaussian generative model 1100. In addition to block 1100, computing environment 1000 includes, for example, computer 1001, wide area network (WAN) 1002, end user device (EUD) 1003, remote server 1004, public cloud 1005, and private cloud 1006. In this embodiment, computer 1001 includes processor set 1010 (including processing circuitry 1020 and cache 1021), communication fabric 1011, volatile memory 1012, persistent storage 1013 (including operating system 1022 and block 1100, as identified above), peripheral device set 1014 (including user interface (UI) device set 1023, storage 1024, and Internet of Things (IoT) sensor set 1025), and network module 1015. Remote server 1004 includes remote database 1030. Public cloud 1005 includes gateway 1040, cloud orchestration module 1041, host physical machine set 1042, virtual machine set 1043, and container set 1044.

COMPUTER 1001 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 1030. As is well understood in the art of computer technology, and depending upon the technology, performance of a computer-implemented method may be distributed among multiple computers and/or between multiple locations. On the other hand, in this presentation of computing environment 1000, detailed discussion is focused on a single computer, specifically computer 1001, to keep the presentation as simple as possible. Computer 1001 may be located in a cloud, even though it is not shown in a cloud in FIG. 10. On the other hand, computer 1001 is not required to be in a cloud except to any extent as may be affirmatively indicated.

PROCESSOR SET 1010 includes one, or more, computer processors of any type now known or to be developed in the future. Processing circuitry 1020 may be distributed over multiple packages, for example, multiple, coordinated integrated circuit chips. Processing circuitry 1020 may implement multiple processor threads and/or multiple processor cores. Cache 1021 is memory that is located in the processor chip package(s) and is typically used for data or code that should be available for rapid access by the threads or cores running on processor set 1010. Cache memories are typically organized into multiple levels depending upon relative proximity to the processing circuitry. Alternatively, some, or all, of the cache for the processor set may be located “off chip.” In some computing environments, processor set 1010 may be designed for working with qubits and performing quantum computing.

Computer readable program instructions are typically loaded onto computer 1001 to cause a series of operational steps to be performed by processor set 1010 of computer 1001 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 1021 and the other storage media discussed below. The program instructions, and associated data, are accessed by processor set 1010 to control and direct performance of the inventive methods. In computing environment 1000, at least some of the instructions for performing the inventive methods may be stored in block 1100 in persistent storage 1013.

COMMUNICATION FABRIC 1011 is the signal conduction path that allows the various components of computer 1001 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 1012 is any type of volatile memory now known or to be developed in the future. Examples include dynamic type random access memory (RAM) or static type RAM. Typically, volatile memory 1012 is characterized by random access, but this is not required unless affirmatively indicated. In computer 1001, the volatile memory 1012 is located in a single package and is internal to computer 1001, but, alternatively or additionally, the volatile memory may be distributed over multiple packages and/or located externally with respect to computer 1001.

PERSISTENT STORAGE 1013 is any form of non-volatile storage for computers that is now known or to be developed in the future. The non-volatility of this storage means that the stored data is maintained regardless of whether power is being supplied to computer 1001 and/or directly to persistent storage 1013. Persistent storage 1013 may be a read only memory (ROM), but typically at least a portion of the persistent storage allows writing of data, deletion of data and re-writing of data. Some familiar forms of persistent storage include magnetic disks and solid state storage devices. Operating system 1022 may take several forms, such as various known proprietary operating systems or open source Portable Operating System Interface-type operating systems that employ a kernel. The code included in block 1100 typically includes at least some of the computer code involved in performing the inventive methods.

PERIPHERAL DEVICE SET 1014 includes the set of peripheral devices of computer 1001. Data communication connections between the peripheral devices and the other components of computer 1001 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 1023 may include components such as a display screen, speaker, microphone, wearable devices (such as goggles and smart watches), keyboard, mouse, printer, touchpad, game controllers, and haptic devices. Storage 1024 is external storage, such as an external hard drive, or insertable storage, such as an SD card. Storage 1024 may be persistent and/or volatile. In some embodiments, storage 1024 may take the form of a quantum computing storage device for storing data in the form of qubits. In embodiments where computer 1001 is required to have a large amount of storage (for example, where computer 1001 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 1015 is the collection of computer software, hardware, and firmware that allows computer 1001 to communicate with other computers through WAN 1002. Network module 1015 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 1015 are performed on the same physical hardware device. In other embodiments (for example, embodiments that utilize software-defined networking (SDN)), the control functions and the forwarding functions of network module 1015 are performed on physically separate devices, such that the control functions manage several different network hardware devices. Computer readable program instructions for performing the inventive methods can typically be downloaded to computer 1001 from an external computer or external storage device through a network adapter card or network interface included in network module 1015.

WAN 1002 is any wide area network (for example, the internet) capable of communicating computer data over non-local distances by any technology for communicating computer data, now known or to be developed in the future. In some embodiments, the WAN 1002 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 1003 is any computer system that is used and controlled by an end user (for example, a customer of an enterprise that operates computer 1001), and may take any of the forms discussed above in connection with computer 1001. EUD 1003 typically receives helpful and useful data from the operations of computer 1001. For example, in a hypothetical case where computer 1001 is designed to provide a recommendation to an end user, this recommendation would typically be communicated from network module 1015 of computer 1001 through WAN 1002 to EUD 1003. In this way, EUD 1003 can display, or otherwise present, the recommendation to an end user. In some embodiments, EUD 1003 may be a client device, such as thin client, heavy client, mainframe computer, desktop computer and so on.

REMOTE SERVER 1004 is any computer system that serves at least some data and/or functionality to computer 1001. Remote server 1004 may be controlled and used by the same entity that operates computer 1001. Remote server 1004 represents the machine(s) that collect and store helpful and useful data for use by other computers, such as computer 1001. For example, in a hypothetical case where computer 1001 is designed and programmed to provide a recommendation based on historical data, then this historical data may be provided to computer 1001 from remote database 1030 of remote server 1004.

PUBLIC CLOUD 1005 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 1005 is performed by the computer hardware and/or software of cloud orchestration module 1041. The computing resources provided by public cloud 1005 are typically implemented by virtual computing environments that run on various computers making up the computers of host physical machine set 1042, which is the universe of physical computers in and/or available to public cloud 1005. The virtual computing environments (VCEs) typically take the form of virtual machines from virtual machine set 1043 and/or containers from container set 1044. It is understood that these VCEs may be stored as images and may be transferred among and between the various physical machine hosts, either as images or after instantiation of the VCE. Cloud orchestration module 1041 manages the transfer and storage of images, deploys new instantiations of VCEs and manages active instantiations of VCE deployments. Gateway 1040 is the collection of computer software, hardware, and firmware that allows public cloud 1005 to communicate through WAN 1002.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 1006 is similar to public cloud 1005, except that the computing resources are only available for use by a single enterprise. While private cloud 1006 is depicted as being in communication with WAN 1002, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 1005 and private cloud 1006 are both part of a larger hybrid cloud.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” are understood to include any integer number greater than or equal to one, i.e. one, two, three, four, etc. The terms “a plurality” are understood to include any integer number greater than or equal to two, i.e. two, three, four, five, etc. The term “connection” can include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

What is claimed is:

1. A computer-implemented method comprising:
using a processor system to encode binary risk factor variables, genotypic risk factor variables, and continuous risk factor variables; and
using the processor system to adversarially train a multivariate Gaussian (MVG) generative model to generate synthetic versions of the binary risk factor variables, synthetic versions of the genotypic risk factor variables, and synthetic versions of the continuous risk factor variables.

2. The computer-implemented method of claim 1 further comprising using the processor system to extract correlations among the synthetic versions of the binary risk factor variables, the synthetic versions of the genotypic risk factor variables, and the synthetic versions of the continuous risk factor variables.

3. The computer-implemented method of claim 1, wherein using the processor system to adversarially train the MVG generative model comprises using a discriminative model of the processor system to adversarially train the MVG generative model.

4. The computer-implemented method of claim 1, wherein:
the synthetic versions of the binary risk factor variables comprise synthetic versions of disease state variables that are either present or not-present;
the synthetic versions of the genotypic risk factor variables comprise synthetic versions of gene mutation state variables; and
the synthetic versions of the continuous risk factor variables comprise synthetic versions of gene expression state variables.

5. The computer-implemented method of claim 1, wherein:
the synthetic versions of the binary risk factor variables fill gaps in a set of non-synthetic binary risk factor variables;
the synthetic versions of the genotypic risk factor variables fill gaps in a set of non-synthetic genotypic risk factor variables; and
the synthetic versions of the continuous risk factor variables fill gaps in a set of non-synthetic continuous risk factor variables.

6. The computer-implemented method of claim 2 further comprising transmitting the synthetic versions of the binary risk factor variables, the synthetic versions of the genotypic risk factor variables, the synthetic versions of the continuous risk factor variables, and correlations among the synthetic versions of the binary risk factor variables, the synthetic versions of the genotypic risk factor variables, and the synthetic versions of the continuous risk factor variables to an omic data analysis system.

7. The computer-implemented method of claim 6, wherein the omic data analysis system comprises a generative adversarial network operable to use the synthetic versions of the binary risk factor variables, the synthetic versions of the genotypic risk factor variables, the synthetic versions of the continuous risk factor variables, and correlations among the synthetic versions of the binary risk factor variables, the synthetic versions of the genotypic risk factor variables, and the synthetic versions of the continuous risk factor variables to generate synthetic portions of diagnostic image data.

8. The computer-implemented method of claim 7, wherein operations performed by the processor system are performed by a cloud computing system.

9. A computer-based system comprising:
a memory; and
a processor system communicatively coupled to the memory;
the processor system configured to perform processor system operations comprising:
encoding binary risk factor variables, genotypic risk factor variables, and continuous risk factor variables; and
adversarially training a multivariate Gaussian (MVG) generative model to generate synthetic versions of the binary risk factor variables, synthetic versions of the genotypic risk factor variables, and synthetic versions of the continuous risk factor variables.

10. The computer-based system of claim 9, wherein the processor system operations further comprise extracting correlations among the synthetic versions of the binary risk factor variables, the synthetic versions of the genotypic risk factor variables, and the synthetic versions of the continuous risk factor variables.

11. The computer-based system of claim 9, wherein adversarially training the MVG generative model comprises using a discriminative model of the processor system to adversarially train the MVG generative model.

12. The computer-based system of claim 9, wherein:
the synthetic versions of the binary risk factor variables comprise synthetic versions of disease state variables that are either present or not-present;
the synthetic versions of the genotypic risk factor variables comprise synthetic versions of gene mutation state variables; and
the synthetic versions of the continuous risk factor variables comprise synthetic versions of gene expression state variables.

13. The computer-based system of claim 9, wherein:
the synthetic versions of the binary risk factor variables fill gaps in a set of non-synthetic binary risk factor variables;
the synthetic versions of the genotypic risk factor variables fill gaps in a set of non-synthetic genotypic risk factor variables; and
the synthetic versions of the continuous risk factor variables fill gaps in a set of non-synthetic continuous risk factor variables.

14. The computer-based system of claim 10, wherein the processor system operations further comprise transmitting the synthetic versions of the binary risk factor variables, the synthetic versions of the genotypic risk factor variables, the synthetic versions of the continuous risk factor variables, and correlations among the synthetic versions of the binary risk factor variables, the synthetic versions of the genotypic risk factor variables, and the synthetic versions of the continuous risk factor variables to an omic data analysis system.

15. The computer-based system of claim 14, wherein the omic data analysis system comprises a generative adversarial network operable to use the synthetic versions of the binary risk factor variables, the synthetic versions of the genotypic risk factor variables, the synthetic versions of the continuous risk factor variables, and correlations among the synthetic versions of the binary risk factor variables, the synthetic versions of the genotypic risk factor variables, and the synthetic versions of the continuous risk factor variables to generate synthetic portions of diagnostic image data.

16. A computer program product comprising a computer readable program stored on a computer readable storage medium, wherein the computer readable program, when executed on a processor system, causes the processor system to perform processor system operations comprising:
encoding binary risk factor variables, genotypic risk factor variables, and continuous risk factor variables; and
adversarially training a multivariate Gaussian (MVG) generative model to generate synthetic versions of the binary risk factor variables, synthetic versions of the genotypic risk factor variables, and synthetic versions of the continuous risk factor variables.

17. The computer program product of claim 16, wherein the processor system operations further comprise extracting correlations among the synthetic versions of the binary risk factor variables, the synthetic versions of the genotypic risk factor variables, and the synthetic versions of the continuous risk factor variables.

18. The computer program product of claim 16, wherein adversarially training the MVG generative model comprises using a discriminative model of the processor system to adversarially train the MVG generative model.

19. The computer program product of claim 17, wherein the processor system operations further comprise transmitting the synthetic versions of the binary risk factor variables, the synthetic versions of the genotypic risk factor variables, the synthetic versions of the continuous risk factor variables, and correlations among the synthetic versions of the binary risk factor variables, the synthetic versions of the genotypic risk factor variables, and the synthetic versions of the continuous risk factor variables to an omic data analysis system.

20. The computer program product of claim 19, wherein the omic data analysis system comprises a generative adversarial network operable to use the synthetic versions of the binary risk factor variables, the synthetic versions of the genotypic risk factor variables, the synthetic versions of the continuous risk factor variables, and correlations among the synthetic versions of the binary risk factor variables, the synthetic versions of the genotypic risk factor variables, and the synthetic versions of the continuous risk factor variables to generate synthetic portions of diagnostic image data.
